(19) United States
(12) Patent Application Publication
(10) Pub. No.: US 2017/0015744 A1
(43) Pub. Date: Jan. 19, 2017

(54) ANTICOAGULANT

(71) Applicants: NATIONAL CEREBRAL AND CARDIOVASCULAR CENTER, Suita-shi (JP); SHINSHU UNIVERSITY, Matsumoto-shi, (JP)

(72) Inventors: Tatsuya SAWAMURA, Suita-shi (JP); Yoshiko FUJITA, Suita-shi (JP); Akemi KAKINO, Suita-shi (JP)

(21) Appl. No.: 15/107,573

(22) PCT Filed: Dec. 23, 2014
(86) PCT No.: PCT/JP2014/084013

§ 371 Date: Sep. 27, 2016

(30) Foreign Application Priority Data
Dec. 27, 2013 (JP) 2013-273308

(51) Int. Cl. C07K 16/28; A61K 31/352

(52) U.S. Cl. CPC C07K 16/28; A61K 31/352; C07K 2317/10; C07K 2317/21; C07K 2317/76; C07K 2317/24

(57) ABSTRACT

The present invention provides an anti-blood coagulant capable of selectively suppressing a pathological blood coagulation reaction without increasing the risk of major bleeding. The anti-blood coagulant in accordance with the present invention includes a binding inhibiting component that inhibits the binding of LOX-1 to at least one of factor V and factor Va.

### TECHNICAL FIELD

The present invention relates to an anti-blood coagulant and a pharmaceutical composition including the anti-blood coagulant.

### BACKGROUND ART

In recent years, as anti-blood coagulants alternative to warfarin, there have been used: argatroban and dabigatran, which are thrombin inhibitors; fondaparinux, which is an indirect factor Xa inhibitor that requires antithrombin III for a cofactor; and rivaroxaban, edoxaban, and apixaban, which are direct factor Xa inhibitors.

Meanwhile, oxidized LDL, which is produced by the oxidization of low-density lipoprotein (hereinafter referred to as “LDL”), is known to function to promote arteriosclerosis, and an anti-atherogenic effect can be brought about by reducing the level of oxidized LDL in blood.

The promoting effect of oxidized LDL on arteriosclerosis is considered to be caused by the uptake of oxidized LDL into vascular endothelial cells through lectin-like oxidized LDL receptor (LOX-1; lectin-like oxidized low-density lipoprotein receptor-1). LOX-1, which was identified as a vascular endothelial oxidized LDL receptor, is currently known as a factor that promotes cardiovascular diseases such as inflammation, arteriosclerosis, thrombus, myocardial infarction, and post-catheterization vascular restenosis. For example, the level of LOX-1 expression is very low in normal blood vessels, but is high in arteriosclerosis nest, catheter-damaged blood vessels, inflamed blood vessels, and the like. Further, soluble LOX-1 in the blood markedly increases especially in acute coronary syndrome and arteriosclerosis obliterans (Non-patent Literature 1).

Patent Literature 1 discloses (i) a DNA sequence consisting essentially of a region coding for a modified low-density lipoprotein receptor of a mammalian vascular endothelial cell, (ii) anti-LOX-1 antibodies, and others.

Patent Literature 2 describes that LOX-1 binds to a blood platelet and is taken into cells and also describes that the binding and cellular uptake of LOX-1 is inhibited by a substance that bind to LOX-1. Further, Patent Literature 3 describes a human monoclonal antibody that binds to human LOX-1.

### CITATION LIST

### Patent Literatures

[Patent Literature 1]
Japanese Patent Application Publication Tokukaihei No. 9-98787 (Publication date: Apr. 15, 1997)
[Patent Literature 2]
Japanese Patent Application Publication Tokukai No. 2000-109435 (Publication date: Apr. 18, 2000)
[Patent Literature 3]
Pamphlet of International publication No. WO 01/064862 (Publication date: Sep. 7, 2001)

### Non-Patent Literature

[Non-patent Literature 1]
Hayashida K et al. Circulation, 112:812-818, 2005

### SUMMARY OF INVENTION

### Technical Problem

Unfortunately, the conventional anti-blood coagulants described above fail to selectively suppress pathological blood coagulation reactions without increasing the risk of major bleeding. In other words, the conventional anti-blood coagulants fail to selectively suppress pathological coagulation reactions that can cause diseases such as thrombosis without suppressing normal coagulation reactions for hemostasis.

The aforementioned drugs, including warfarin, inhibit the most essential protease in the protease cascade of a blood coagulation system. Those drugs have a very strong coagulation suppressing ability, but involve the risk of causing major bleeding at the same time. Such a side effect is inextricably linked with the blood coagulation suppressing effect, which is an inherent effect of the drugs. As such, it is difficult to prevent the side effect. It has been reported that the newly developed thrombin inhibitors and factor Xa inhibitors cause a lower incidence of major bleeding than warfarin (Connolly S J, N. Engl. J. Med., 363:1875-1876, 2010; Patel M R, N. Engl. J. Med., 365:883-891, 2011; Granger C B, N. Engl. J. Med., 365:981-992, 2011). Nevertheless, it is a reality that attention centers on the development of inhibitors to prevent the side effect as opposed to the factor Xa inhibitors (Lu G, Nature Med., 19:446-451, 2013).

The present invention has been attained in view of the above-described problems of the conventional anti-blood coagulants, and it is an object of the present invention to provide an anti-blood coagulant that selectively suppresses a pathological blood coagulation reaction without increasing the risk of major bleeding.

### Solution to Problem

The inventors of the present invention diligently studied to solve the foregoing problems, and surprisingly obtained an entirely new finding that LOX-1 can bind to factor V or factor Va to form a complex and thus leads to promotion of blood coagulation reaction. On the basis of such a new finding, the inventors of the present invention found that inhibition of the binding LOX-1 to factor V or factor Va allows a blood coagulation reaction for which LOX-1 is responsible to be selectively suppressed while maintaining a blood coagulation cascade for which LOX-1 is not responsible, and finally accomplished the present invention. That is, the present invention encompasses the following inventions:

In order to solve the foregoing problems, an anti-blood coagulant in accordance with the present invention includes a binding inhibiting component that inhibits the binding of LOX-1 to at least one of factor V and factor Va.

### Advantageous Effects of Invention

Unlike the conventional anti-blood coagulants that target thrombin or factor Xa, the present invention inhibits the binding of LOX-1 to factor V or factor Va. This makes it possible to selectively suppress a blood coagulation reaction for which LOX-1 is responsible while maintaining a blood coagulation cascade for which LOX-1 is not responsible. This brings about the effect of selectively suppressing a pathological blood coagulation reaction without increasing the risk of major bleeding.

### BRIEF DESCRIPTION OF DRAWINGS

FIG. 1 is a view illustrating a cascade of a blood coagulation system.

FIG. 2 is a diagram illustrating the structure of factor V.

FIG. 3 is a view showing results obtained by running LOX-1 binding proteins on SDS-PAGE.

FIG. 4 is a set of graphs showing results of evaluation of binding affinity of LOX-1 to factor V and factor Va by surface plasmon resonance. (a) of FIG. 4 shows results for factor V, wherein a sensorgram indicated by (i) represents a response on the vertical axis and a time on the horizontal axis, while a graph indicated by (ii) represents an equilibrium binding amount Req on the vertical axis and a concentration on the horizontal axis. (b) of FIG. 4 shows results for factor Va, wherein a sensorgram indicated by (i) represents a response on the vertical axis and a time on the horizontal axis, while a graph indicated by (ii) represents an equilibrium binding amount Req on the vertical axis and a concentration on the horizontal axis.

FIG. 5 is a graph showing results of evaluation of inhibitory effects of anti-LOX-1 antibodies on binding of LOX-1 to factor V and factor Va by LOX-1 immobilization ELISA.

FIG. 6 is a view showing results of detection of a LOX-1 binding site of factor Va with use of an anti-L chain antibody and an anti-H chain antibody. (a) of FIG. 6 shows a result obtained for a well to which an anti-L chain antibody and a CaCl₂ solution were added, and (b) of FIG. 6 shows a result obtained for a well to which the anti-L chain antibody and EDTA were added, and (c) of FIG. 6 shows a result obtained for a well to which an anti-H chain antibody and the CaCl₂ solution were added, and (d) of FIG. 6 shows a result obtained for a well to which the anti-H chain antibody and EDTA were added.

(a) of FIG. 7 is a graph showing results of evaluation of binding of LOX-1 to each domain of a H chain of factor V. (b) of FIG. 7 is a graph showing results of evaluation of inhibitory effects of anti-LOX-1 antibodies on binding of LOX-1 to each domain of a H chain of factor V.

(a) of FIG. 8 is a graph showing blood coagulation times that vary depending on the concentration of LOX-1. (b) of FIG. 8 is a graph showing blood coagulation times of the cases in which each antibody and LOX-1 were mixed.

FIG. 9 is a view showing results of a recalcification blood coagulation test using factor V-deficient blood plasma.

FIG. 10 is a graph showing results obtained by adding procyanidin to LOX-1.

### DESCRIPTION OF EMBODIMENTS

The present invention will be described in detail below. The scope of the present invention is not bound by the descriptions that follow, and embodiments and modifications other than those illustrated below may be implemented as appropriate to such an extent that the gist of the present invention is not defeated. All of the known documents described herein are incorporated herein by reference.

[Anticoagulant of the Present Invention]

An anti-blood coagulant of the present invention includes a binding inhibiting component that inhibits the binding of LOX-1 to at least one of factor V and factor Va.

The inventors of the present invention first found that LOX-1 binds to factor V or factor Va to form a complex and thus leads to promotion of coagulation reaction. On the basis of such a whole new finding, the inventors of the present invention found that it is possible to achieve an anti-blood coagulant that inhibits LOX-1 from binding to factor V or factor Va, thereby allowing a blood coagulation reaction for which LOX-1 is responsible to be suppressed while maintaining a blood coagulation cascade for which LOX-1 is not responsible.

As described earlier, the conventional anti-blood coagulants, including warfarin, the thrombin inhibitors, and the factor Xa inhibitors, have functionally inseparable effects: the main effect (coagulation suppression) and the side effect (bleeding). As such, the conventional anti-blood coagulants have difficulty balancing the merit of preventing thrombosis and the risk of bleeding when they are used. Therefore, the conventional anti-blood coagulants are capable of preventing thrombosis, but always entail the problem that they may lead to bleeding death.

A solution to the above problems is limited to, for example, control of dosages of the drugs. The present invention allows for an approach different in qualitative terms from those of the conventional anti-blood coagulants, and enables curbing a pathological coagulation reaction to suppress thrombosis, instead of completely curbing the blood coagulation effect (that is, without increasing the risk of bleeding).

The present invention, unlike the conventional anti-blood coagulants, does not intend to target protease or any blood coagulation factor. Instead, the present invention intends to target LOX-1, which is a regulatory molecule that regulates activities of the protease or the blood coagulation factor. Therefore, the anti-blood coagulant of the present invention is considered to exert an anti-blood coagulation effect only in a case where LOX-1 is functioning pathologically, without excessively suppressing a coagulation function.

In addition, a factor-X or thrombin knockout mouse is lethal, whereas a LOX-1 knockout mouse normally develops and grows (Mehta et al. Circulation Research 100: 1634-1642, 2007). From that fact, it can be presumed that the present invention has safety.

Furthermore, in terms of pharmacokinetics, the anti-blood coagulant of the present invention, which targets LOX-1, is not affected by ingested food, unlike warfarin, and is not affected by a liver function and a renal function, unlike a low molecular weight thrombin inhibitor and a factor Xa inhibitor. Moreover, the anti-blood coagulant of the present invention is less likely to cause drug interactions since it will not become a substrate for P-glycoprotein or cytochrome P450 (CYP).

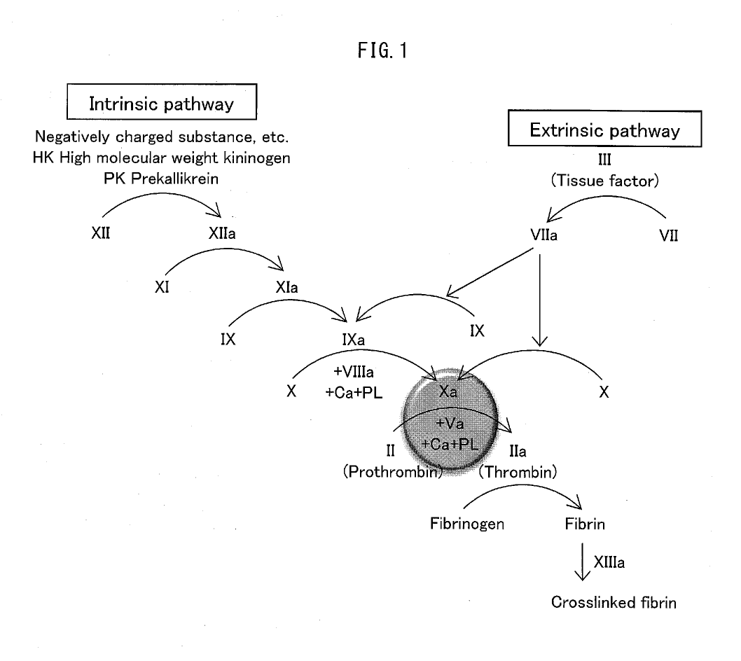

Factor V (and factor Va, which is a factor produced by activating factor V) is a kind of blood coagulation factor. FIG. 1 is a view illustrating a cascade of a blood coagulation system. To obtain an anti-blood coagulation effect, it is necessary to inhibit a part of a cascade which part is located downstream from a confluence of an intrinsic pathway and an extrinsic pathway in the blood coagulation system. Factor V (factor Va) is a coenzyme that acts in the part of the cascade which part is located downstream from the confluence (i.e., factor Xa) of the intrinsic pathway and the extrinsic pathway in the blood coagulation system. As such, by inhibiting the binding of LOX-1 to factor V or factor Va, it is possible to suppress blood coagulation.

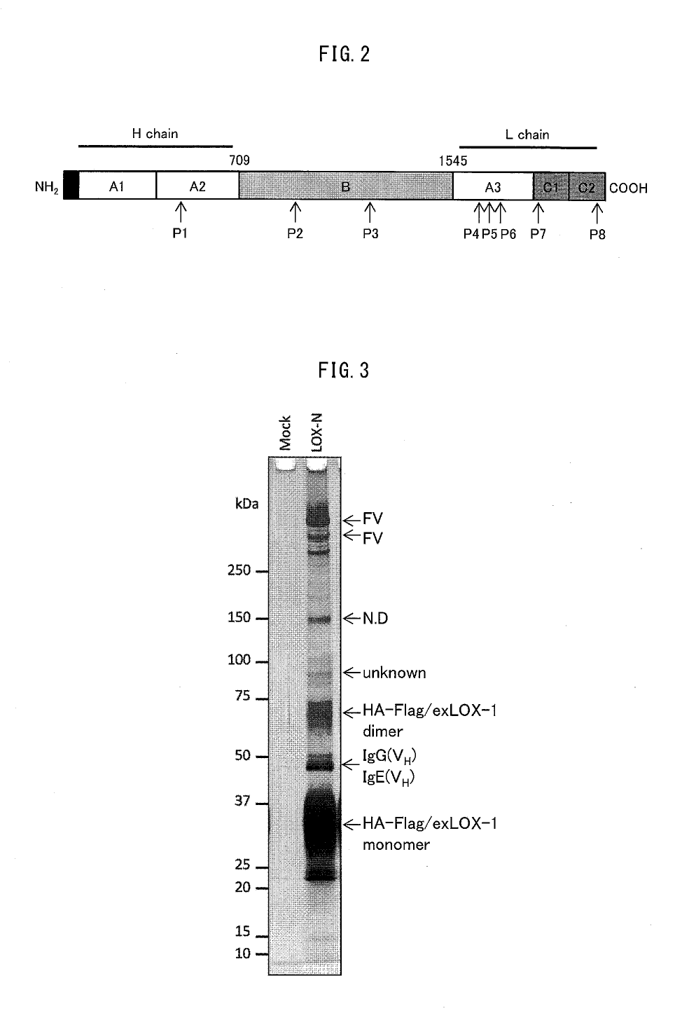

FIG. 2 is a view illustrating the structure of the factor V. The factor V is a protein that contains an H chain and an L chain. The factor V is composed of A1, A2, B, A3, C1, and C2 domains, in order of proximity to the N-terminus. The A1 domain and A2 domain form the H chain, while the A3 domain, the C1 domain, and the C2 domain form the L chain. The factor V, when activated, is separated into the H chain and the L chain to produce factors Va. Note that the inventors of the present invention showed, as described later in Example 5, that the A2 domain binds to LOX-1.

The factor V can also be referred herein to as “FV”. Similarly, the factor Va can also be referred to as “FVa”. Further, the H chain, which is also called heavy chain, can also be abbreviated herein as “HC”. Still further, the L chain, which is also called light chain, can also be abbreviated herein as “LC”.

Whether the binding inhibiting component inhibits the binding of LOX-1 to the factor V or the factor Va can be confirmed by a method described in section “Examples”. Specifically, it can be confirmed by the method described in Example 3. For example, when it is found by the method described in Example 3 that there is a significant decrease in the binding of LOX-1 to the factor V in the presence of the binding inhibiting component as compared with the binding of LOX-1 to the factor V in the absence of the binding inhibiting component, the binding inhibiting component can be determined to inhibit the binding of LOX-1 to the factor V. This also applies to the binding of LOX-1 to the factor Va.

The anti-blood coagulation effect can be confirmed by, for example, a recalcification blood coagulation test, which will be described in Examples 7 to 9. In a case where the recalcification blood coagulation test performed in the presence of LOX-1 and factor V shows that there is a significant increase in a blood coagulation time measured in the presence of the binding inhibiting component as compared to a blood coagulation time measured in the absence of the binding inhibiting component, it is possible to determine that the binding inhibiting component exhibits the anti-blood coagulation effect. Further, in a case where a blood coagulation time measured in the presence of LOX-1, factor V, and the binding inhibiting component is on a par with a blood coagulation time measured in the absence of LOX-1 and the binding inhibiting component, it is possible to determine that the binding inhibiting component does not increase the risk of bleeding.

Still further, in a case where (i) a blood coagulation time measured in the absence of LOX-1 and in the presence of the binding inhibiting component is on a par with a blood coagulation time measured in the absence of LOX-1 and the binding inhibiting component, and (ii) there is a significant increase in a blood coagulation time measured in the presence of LOX-1 and the binding inhibiting component as compared to a blood coagulation time measured in the presence of LOX-1 and in the absence of the binding inhibiting component, it is possible to determine that the binding inhibiting component is capable of suppressing a blood coagulation reaction for which LOX-1 is responsible, while maintaining a blood coagulation cascade for which LOX-1 is not responsible.

It should be noted that when, for example, a Student's t-test conducted on the pieces of data shows that there is a significant decrease or increase in one data as compared with the other data at a level of significance of less than 5%, it can be determined that “there is a significant decrease” or “there is a significant increase”.

The binding inhibiting component is not limited to a specific substance, provided that it can bind to LOX-1 to inhibit the binding of LOX-1 to the factor V. Examples of the binding inhibiting component include an anti-LOX-1 antibody and polyphenol.

As used herein, the term “antibody” refers to an immunoglobulin (IgA, IgD, IgE, IgG, and IgM; and Fab fragments of IgA, IgD, IgE, IgG, and IgM, F(ab′)₂ fragments thereof, and Fc fragments thereof), and non-limiting examples of the “antibody” include a polyclonal antibody, a monoclonal antibody, a single-chain antibody, an anti-idiotype antibody, and a humanized antibody.

The “antibody” can be prepared in accordance with any of various known methods (e.g., the methods described in HarLow et al., “Antibodies: A Laboratory Manual, Cold Spring Harbor Laboratory”, New York (1988) and Iwasaki et al., “Monoclonal Antibody: Hybridoma and ELISA”, KODANSHA (1991)).

The monoclonal antibody can be prepared by using a well-known method in the art (Refer to, for example, a hybridoma method (Kohler, G. and Milstein, C., Nature 256, 495-497 (1975)), a trioma method, a human B-cell hybridoma method (Kozbor, Immunology Today 4, 72 (1983)) and an EBV-hybridoma method (Monoclonal Antibodies and Cancer Therapy, Alan R Liss, Inc., 77-96 (1985))).

It is clear that Fab and F(ab′)₂ fragments and other fragments of the above-described antibodies can be used in accordance with the method(s) disclosed herein. Those fragments are produced by proteolytic cleavage using an enzyme that is typified by papain, which produces Fab fragments, and pepsin, which produces (F(ab′)₂ fragments. Alternatively, the fragments that bind to LOX-1 may be produced by applying a recombinant DNA technology or by synthetic chemistry.

In other words, the antibody that is usable in the present invention include at least an antibody fragment that recognizes LOX-1 and inhibits an activity of LOX-1 (e.g., Fab fragment and F(ab′)₂ fragment). Such an antibody does not require, for example, a specific type of immunoglobulin (IgA, IgD, IgE, IgY, IgG, or IgM), a specific chimeric antibody preparation method, a specific peptide antigen preparation method, and others. Therefore, antibodies obtained by methods other than the above-described methods are also usable in the present invention.

Examples of the polyphenol that is usable as the binding inhibiting component include flavonoids. Examples of the flavonoids include procyanidin. It is known that procyanidin inhibits the binding of LOX-1 to oxidized LDL (Nishizuka et al., Proc Jpn Acad Ser B Phys Biol Sci, 87: 104-113, 2011). However, it was first found by the inventors of the present invention that procyanidin has an anti-blood coagulation effect, as shown in Example 9.

The amount of the binding inhibiting component contained in the anti-blood coagulant of the present invention is not particularly limited, provided that it is a sufficient amount to bring about the effects of inhibiting the binding of LOX-1 to the factor V or the factor Va, and can be determined as appropriate in consideration of the purity, dosage form, or method of intake of the binding inhibiting component. For example, it is preferable that the binding inhibiting component be contained in a percentage of 50% (w/w) or higher, more preferably 80% (w/w) or higher, even more preferably 100% (w/w), of the anti-blood coagulant.

[Pharmaceutical Composition of the Present Invention]

A pharmaceutical composition of the present invention contains the aforementioned anti-blood coagulant of the present invention. The pharmaceutical composition of the present invention inhibits the binding of LOX-1 to the factor V or the factor Va, thereby being capable of suppressing the blood coagulation reaction for which LOX-1 is responsible, while keeping the blood coagulation cascade for which LOX-1 is not responsible. As such, the pharmaceutical composition of the present invention is expected to be effective in preventing thrombosis.

The pharmaceutical composition of the present invention may further contain a pharmaceutically acceptable component in addition to the anti-blood coagulant (e.g., a pharmaceutically acceptable carrier, etc.), provided the activity of inhibiting the binding of LOX-1 to the factor V or the factor Va is not impaired.

The “pharmaceutically acceptable carrier” is described here. As used herein, the term “pharmaceutically acceptable carrier” (hereinafter referred to simply as “carrier”) refers to a substance that is used for the purpose of aiding in drug formulation in the manufacture of a medicine or agricultural chemicals such as animal drugs and that does not have a deleterious effect on the binding inhibiting component. Furthermore, the term is also intended to mean that there are no toxic consequences in an individual given the pharmaceutical composition of the present invention and that the carrier per se does not induce the production of a hazardous antibody.

The carrier can be selected as appropriate from among various organic or inorganic carrier substances that are usable as raw materials for drug formulation, depending on the below-mentioned forms of administration or dosage forms of the pharmaceutical composition. For example, the carrier may be combined as any of the following: an excipient, a lubricant, a binder, disintegrant etc. in a solid preparation; a solvent, a solubilizer, a suspension, a tonicity agent, a buffer, a soothing agent, etc. in a liquid preparation; an antiseptic; an antioxidant; a stabilizer; a flavoring agent; and the like. However, the present invention is not limited to these examples.

Examples of the “excipient” include lactose, saccharose, D-mannitol, xylitol, sorbitol, erythritol, starch, crystalline cellulose, etc. However, the “excipient” is not limited to a specific one, provided that it is an excipient normally employed in the field of pharmaceuticals.

Examples of the “lubricant” include magnesium stearate, calcium stearate, wax, talc, colloid silica, etc. However, the “lubricant” is not limited to a specific one, provided that it is a lubricant normally employed in the field of pharmaceuticals.

Examples of the “binder” include alpha starch, methyl cellulose, crystalline cellulose, saccharose, D-mannitol, trehalose, dextrin, hydroxypropylcellulose, hydroxypropyl methylcellulose, polyvinyl pyrrolidone, etc. However, the “binder” is not limited to a specific one, provided that it is a binder normally employed in the field of pharmaceuticals.

Examples of the “disintegrant” include starch, carboxymethyl cellulose, low substituted hydroxypropylcellulose, carboxymethylcellulose calcium, croscarmellose sodium, carboxymethyl starch sodium, etc. However, the “disintegrant” is not limited to a specific one, provided that it is a disintegrant normally employed in the field of pharmaceuticals.

Examples of the “solvent” include water for injection, alcohol, propylene glycol, macrogol, sesame oil, corn oil, tricaprilin, etc. However, the “solvent” is not limited to a specific one, provided that it is a solvent normally employed in the field of pharmaceuticals.

Examples of the “solubilizer” include polyethylene glycol, propylene glycol, D-mannitol, trehalose, benzyl benzoate, ethanol, trisaminomethane, cholesterol, triethanolamine, sodium carbonate, sodium citrate, etc. However, the “solubilizer” is not limited to a specific one, provided that it is a solubilizer normally employed in the field of pharmaceuticals.

Examples of the “suspension” include: surface-active agents, such as stearyl triethanolamine, sodium lauryl sulfate, lauraminopropionic acid, lecithin, benzalkonium chloride, benzethonium chloride, and glyceryl monostearate; hydrophilic polymers, such as polyvinyl alcohol, polyvinyl pyrrolidone, carboxymethylcellulose sodium, methyl cellulose, hydroxymethyl cellulose, and hydroxypropyl cellulose; and the like. However, the “suspension” is not limited to a specific one, provided that it is a suspension normally employed in the field of pharmaceuticals.

Examples of the “tonicity agent” include sodium chloride, glycerin, D-mannitol, etc. However, the “tonicity agent” is not limited to a specific one, provided that it is a tonicity agent normally employed in the field of pharmaceuticals.

Examples of the “buffer” include: buffers such as phosphate, acetate, carbonate, and citrate; and the like. However, the “buffer” is not limited to a specific one, provided that it is a buffer normally employed in the field of pharmaceuticals.

Examples of the “soothing agent” include benzyl alcohol etc. However, the “soothing agent” is not limited to a specific one, provided that it is a soothing agent normally employed in the field of pharmaceuticals.

Examples of the “antiseptic” include p-hydroxybenzoic acid esters, chlorobutanol, benzyl alcohol, phenethyl alcohol, dehydroacetic acid, sorbic acid, etc. However, the “antiseptic” is not limited to a specific one, provided that it is an antiseptic normally employed in the field of pharmaceuticals.

Examples of the “antioxidant” include, sulfite, ascorbic acid, etc. However, the “antioxidant” is not limited to a specific one, provided that it is an antioxidant normally employed in the field of pharmaceuticals.

Further, the stabilizer and the flavoring agent are not limited to specific ones, provided that they are those normally employed in the field of pharmaceuticals.

A form of administration of the pharmaceutical composition in accordance with the present invention may be oral or parenteral (e.g., intravenous, rectal, interperitoneal, intramuscular, or subcutaneous), and any suitable route of administration can be employed for any form of drug formulation. As used herein, the term “parenteral” refers to any mode of administration including intraventricular injections and infusions, intravenous injections and infusions, intramuscular injections and infusions, interperitoneal injections and infusions, intrasternal injections and infusions, subcutaneous injections and infusions, and intraarticular injections and infusions.

In a case where the pharmaceutical composition in accordance with the present invention is administered orally, examples of dosage forms of such a pharmaceutical composition (hereinafter also referred to as “oral drug”) include: a solid preparation, such as a powdered medicine, granules, a tablet, a liposome, or a capsule (including a soft capsule and a microcapsule), and a powdered drug; and a liquid preparation such as a syrup.

The “liquid preparation” can be prepared with the carrier by a method normally used in the field of pharmaceuticals. Examples of the carrier here include: water; an organic solvent such as glycerol, glycol, polyethylene glycol; a mixture of water and any of these organic solvents; and the like. Further, the liquid preparation may further contain a solubilizer, a buffer, a tonicity agent, a stabilizer, and/or the like.

The “solid preparation” can be prepared with the carrier by a method normally used in the field of pharmaceuticals. Examples of the carrier here include an excipient, a lubricant, a binder, a disintegrant, a stabilizer, a flavoring agent, and/or the like.

In the preparation of such an oral drug, a lubricant, a glidant, a colorant, perfume, and/or the like may be further combined therewith for any purpose.

Alternatively, in a case where the pharmaceutical composition in accordance with the present invention is administered parenterally, examples of dosage forms of such a pharmaceutical composition (hereinafter also referred to as “parenteral drug”) include injections, suppositories, pellets, drops, etc. Such a parenteral drug can be prepared by, in a manner known in the field of pharmaceuticals, dissolving or suspending the pharmaceutical composition in accordance with the present invention in a diluent (e.g., distilled water for injection, a physiological saline, an aqueous solution of glucose, vegetable oil for injection, sesame oil, peanut oil, soy bean oil, corn oil, propylene glycol, polyethylene glycol, etc.) and further adding a bactericide, a stabilizer, a tonicity agent, a soothing agent, and/or the like for any purpose.

An embodiment of the pharmaceutical composition in accordance with the present invention may be a sustained-release preparation prepared by a technique normally employed in the field of pharmaceuticals.

The pharmaceutical composition in accordance with the present invention may be administered alone or in combination with another drug. Examples of the administration of the pharmaceutical composition in combination with another drug include simultaneous administration of the pharmaceutical composition as a mixture with another drug, simultaneous or concurrent administration of the pharmaceutical composition as a drug separate from another drug, and administration of the pharmaceutical composition over time. However, the present invention is not limited to these examples.

Further, the number of doses of the pharmaceutical composition in accordance with the present invention is administered per day is not particularly limited. The pharmaceutical composition in accordance with the present invention may be administered in a single dose per day or in multiple doses per day, provided that the anti-blood coagulant of the present invention is administered within a required daily dose range.

A person skilled in the art will easily understand that the pharmaceutical composition of the present invention can be applied to non-human mammals (e.g., mice, rats, rabbits, dogs, cats, cows, horses, pigs, monkeys, etc.), as well as humans.

The amount of the anti-blood coagulant (or the binding inhibiting component) in the pharmaceutical composition of the present invention is not limited to a specific amount, provided that it is a sufficient amount to bring about the effect of inhibiting the binding of LOX-1 to the factor V, and can be determined as appropriate in consideration of the purity, dosage form, or method of intake of the binding inhibiting component and the sex, age, body weight, health condition, etc. of a subject who takes in the pharmaceutical composition.

In order to bring about the effect of an activity of inhibiting the binding of LOX-1 to the factor V or the factor Va, the binding inhibiting component can be contained in the pharmaceutical composition of the present invention so that an intake of the binding inhibiting component is 10 μg to 1000 mg (alternatively, 10 μg to 500 mg) per administration per adult per day in dry weight.

Especially, in a case where a dosage form of the pharmaceutical composition of the present invention is an injection, the injection can be manufactured by dissolving or suspending the pharmaceutical composition in a pharmaceutically acceptable nontoxic carrier such as, for example, a physiological saline or a commercially available distilled water for injection, so that an antibody concentration is from 0.1 μg/mL (antibody/carrier) to 10 mg/mL (antibody/carrier).

The injection thus manufactured can be administered to human patients in need of a treatment at a rate of 1 μg to 100 mg per kilogram of body weight, preferably at a rate of 50 μg to 50 mg per kilogram of body weight, per administration once to several times per day.

The anti-blood coagulant and pharmaceutical composition of the present invention can be used to practice anti-blood coagulant therapy on, for example, a patient in need of thrombosis prevention. Examples of the patient in need of the thrombosis prevention include patients with acute cerebral infarction, individuals at high risk for cerebral infarction, post-PCI patients, individuals at high risk for myocardial infarction, patients with arteriosclerosis obliterans, patients with systemic inflammatory response syndrome, patients with disseminated intravascular coagulation syndrome, and post-orthopedic surgery patients.

The present invention is not limited to the embodiments, but can be altered by a skilled person in the art within the scope of the claims. An embodiment derived from a proper combination of technical means each disclosed in a different embodiment is also encompassed in the technical scope of the present invention.

The present invention can be configured as follows:

In order to solve the above problems, an anti-blood coagulant in accordance with the present invention includes a binding inhibiting component that inhibits binding of LOX-1 to at least one of factor V and factor Va.

As described earlier, it is known that the level of LOX-1 expression is very low in normal blood vessels, but is high in arteriosclerosis nest, catheter-damaged blood vessels, inflamed blood vessels, and the like. Therefore, LOX-1 is considered to promote blood coagulation reactions primarily in the above-described pathological blood vessels, which are different from normal blood vessels, and pathological conditions.

The anti-blood coagulant in accordance with the present invention is capable of selectively suppressing a blood coagulation reaction for which LOX-1 is responsible while maintaining a blood coagulation cascade for which LOX-1 is not responsible. This makes it possible to selectively suppress a pathological blood coagulation reaction without increasing the risk of major bleeding.

The anti-blood coagulant in accordance with the present invention may be a binding inhibiting component that inhibits binding of LOX-1 to an H chain of at least one of factor V and factor Va.

The anti-blood coagulant in accordance with the present invention may be a binding inhibiting component that inhibits binding of LOX-1 to A2 domain of at least one of factor V and factor Va.

The binding inhibiting component may be an anti-LOX-1 antibody.

The binding inhibiting component may be polyphenol.

Further, the present invention encompasses a pharmaceutical composition including an anti-blood coagulant in accordance with the present invention.

Incidentally, Patent Literature 1 fails to study suppression of a blood coagulation reaction and merely discloses that an anti-LOX-1 antibody which can be used for diagnosis can be prepared with use of LOX-1.

Patent Literature 2 confirms an inhibitory effect of an anti-LOX-1 antibody on the binding of platelets to LOX-1. Meanwhile, Patent Literature 3 confirms an inhibitory effect of an anti-LOX-1 antibody on cellular uptake of oxidized LDL, a suppression effect of an anti-LOX-1 antibody on the formulation of thrombus in an artery, and other effect. The techniques described in Patent Literatures 2 and 3 are primarily based on the inhibitory effect on the binding of platelets to LOX-1 and are considered to exert an antiplatelet effect.

Here, an anti-platelet agent is different from an anti-blood coagulant (anticoagulant). Specifically, the anti-platelet agent inhibits the aggregation of platelets to prevent the formation of white thrombus primarily in an artery, whereas the anti-blood coagulant inhibits the formation of fibrin to prevent the formation of red thrombus primarily in a vein (free encyclopedia, Wikipedia, “an anti-platelet agent”, [online], [searched on Oct. 15, 2013], Internet <http://ja.wikipedia.org/wiki/%E6%8A%97%E8%A1%80%E5%B0%8F%E6%9D%BF%E5%89%A4>; and RIKAGAKUJITEN, 4th Ed., p. 418, IWANAMI SHOTEN).

Therefore, the anti-blood coagulant in accordance with the present invention is totally different from the techniques described in Patent Literatures 1 to 3.

### EXAMPLES

The embodiments of the present invention are described in more detail with reference to the Examples below. Of course, the present invention is not limited to the Examples below, and there are lots of details in various aspects. In the Examples below, the term “significantly” refers to a case where the Student's t-test showed a significant difference at a level of significance of less than 5%. In the drawings, an asterisk (*) denotes a case where there was a significant difference at a level of significance of less than 5%.

### Example 1

### Identification of LOX-1 Binding Proteins in Blood Plasma

Among proteins contained in blood plasma, proteins that bind to LOX-1 (hereinafter referred to as “LOX-1 binding proteins”) were identified.

Epitope-tagged recombinant LOX-1 proteins were prepared by the following method. First, LOX-1 plasmid (containing NM002543 (61-273, aa), pDisplay, and Flag therein) was transfected into FreeStyle293 cells. Subsequently, LOX-1 proteins secreted by the FreeStyle293 cells during the culture were collected, purified with use of ANTI-FLAG M2 affinity gel (Sigma-Aldrich), and then eluted with FLAG Peptide (Sigma-Aldrich) for use.

Further, human blood plasma was obtained by drawing blood with use of EDTA. The epitope-tagged recombinant LOX-1 proteins were mixed with the human blood plasma, and the mixture thus obtained was subjected to immunoprecipitation with a tag-recognizing antibody. LOX-1 binding proteins obtained by purification were run on SDS-PAGE and then silver-stained. Thereafter, MS/MS analysis was performed for analysis of amino acid sequences to identify the LOX-1 binding proteins.

FIG. 3 shows results obtained by running the LOX-1 binding proteins on SDS-PAGE. As a result of MS/MS analysis, the amino acid sequences of SEQ ID NO: 1 to SEQ ID NO: 8 shown in Table 1 were obtained. The results of the MS/MS analysis showed that the factor V, which is the blood coagulation factor, was contained in the LOX-1 binding proteins. Note that P1 to P8 in Table 1 correspond to P1 to P8 in FIG. 2, respectively.

TABLE 1

| | | Amino acid |
|---|---|---|
| Protein | No. | sequences obtained by MS/MS analysis |
| Factor V | P1 | ⁴⁷³R.AVQPGETYTYK.W⁴⁸⁵ |
| | | (SEQ ID NO: 1) |
| | P2 | ⁸⁴⁸R.LLSLGAGEFR.S⁸⁵⁹ |
| | | (SEQ ID NO: 2) |
| | P3 | ¹⁰²²K.SQFLIKTR. K¹⁰³¹ |
| | | (SEQ ID NO: 3) |
| | P4 | ¹⁵⁵⁹R.ETDIEDSDDIPEDTTYKK.V¹⁶¹⁸ |
| | | (SEQ ID NO: 4) |
| | P5 | ¹⁶²¹R.KYLDSTFTK.R¹⁶³¹ |
| | | (SEQ ID NO: 5) |
| | P6 | ¹⁶⁴⁹R.AEVDDVIQVRFK.N¹⁶⁶² |
| | | (SEQ ID NO: 6) |
| | P7 | ¹⁸⁹³R.AGMQTPFLIMDRDCR.M¹⁹⁰⁹ |
| | | (SEQ ID NO: 7) |
| | P8 | ²¹⁷⁰K.SSMVDKIFEGNTNTK.G²¹⁸⁶ |
| | | (SEQ ID NO: 8) |

### Example 2

### Evaluation of Binding Affinity of LOX-1 to FV and FVa by Surface Plasmon Resonance

By surface plasmon resonance, binding affinity of LOX-1 protein to FV and FVa was evaluated. The LOX-1 proteins were prepared by the following method. First, LOX-1 plasmid (containing NM002543 (61-273, aa), pcDNA4, 6×His, Igκ secretion signal therein) was transfected into FreeStyle293 cells. Note that the Igκ secretion signal was obtained by pSecTag/FRT/V5-HisTOPO (Invitrogen). Subsequently, LOX-1 proteins secreted by the FreeStyle293 cells during the culture were collected and then prepared by His-tag purification with TALON Metal Affinity Resin (TAKARA). As a result, epitope-tagged recombinant LOX-1 proteins were prepared. FV and FVa were purchased from Haematologic Technologies Inc. (Human Factor V: HCV-0100 was used as FV, and Human Factor Va: HCVA-0110 were used as FVa). The surface plasmon resonance was performed using Biacore (manufactured by GE Healthcare, Inc.). A sensor chip having FV immobilized thereon and a sensor chip having FVa immobilized thereon were prepared, and the LOX-1 proteins of 0 nM to 400 nM were flown at a flow rate of 10 μL/min.

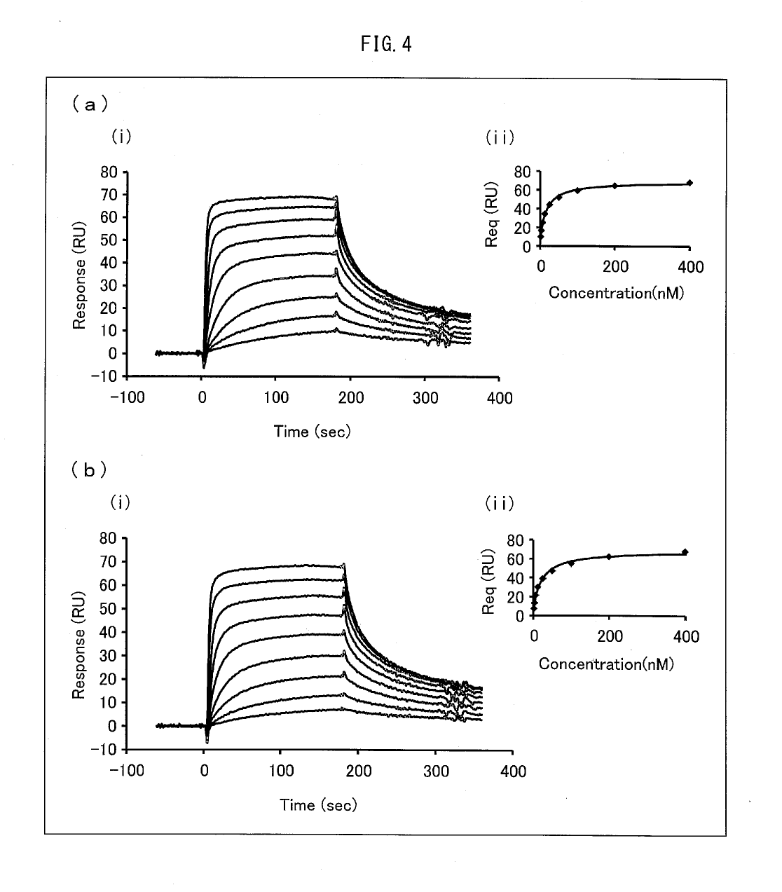

Obtained results are shown in FIG. 4. (a) of FIG. 4 shows results for FV, wherein a sensorgram indicated by (i) represents a response on the vertical axis and a time on the horizontal axis, while a graph indicated by (ii) represents an equilibrium binding amount Req on the vertical axis and a concentration on the horizontal axis. Dissociation constant K_{D} calculated from those results was 15.25±1.28 nM. Further, (b) of FIG. 4 shows results for FVa, wherein a sensorgram indicated by (i) represents a response on the vertical axis and a time on the horizontal axis, while a graph indicated by (ii) represents an equilibrium binding amount Req on the vertical axis and a concentration on the horizontal axis. Dissociation constant K_{D} calculated from those results was 22.14±0.12 nM.

From this result, it is determined that the LOX-1 protein very strongly binds to FV and FVa.

### Example 3

### Evaluation of Inhibitory Effects of Anti-LOX-1 Antibodies on Binding of LOX-1 to FV and FVa (LOX-1 Immobilization ELISA)

The activity of binding LOX-1 to FV and FVa was evaluated by Enzyme Linked Immunosorbent Assay (ELISA). ELISA was performed in the following manner. On a 384-well plate (manufactured by Greiner), LOX-1 (5 μg/mL for each well) was immobilized at 4° C. overnight. After blocked with a solution containing 3% BSA/10 mM HEPES and 150 mM NaCl at pH7.4, the plate was washed twice with PBS (−). To the plate were added anti-LOX-1 antibodies or IgG, after which the plate was pre-incubated. After the plate was washed twice with PBS (−), FV or FVa was added to each well and was then reacted for 1 hour at room temperature. As controls, a well in which LOX-1 was not immobilized and a well to which neither any anti-LOX-1 antibody nor IgG were added, were also prepared. As the anti-LOX-1 antibodies, HUC348 (Antibody 1), HUC52 (Antibody 2), and HUC52 humanized antibody (Antibody 3) were used.

Detection of the activity of binding LOX-1 to FV and FVa was performed with use of an anti-FVL chain antibody, a biotin-labeled anti-mouse IgG antibody, Horseradish peroxidase streptavidin, and a TMB reagent. Then, absorbance at 450 nm was measured. For FVa, ELISA was also performed in a similar manner.

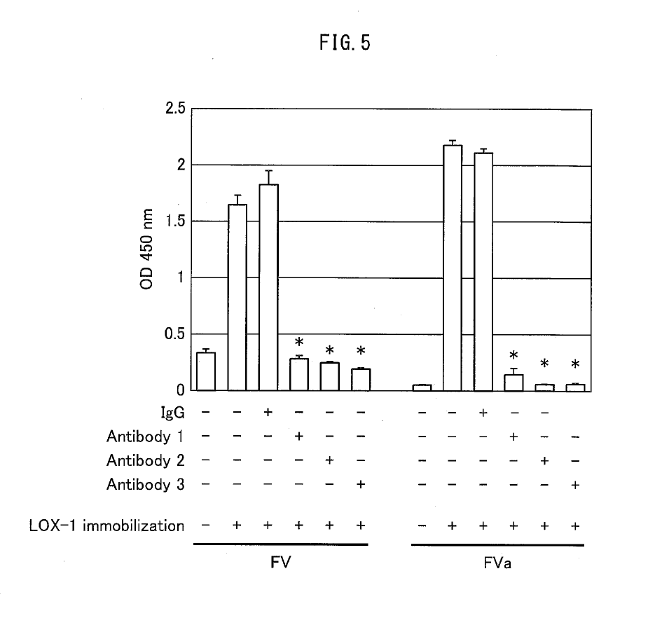

FIG. 5 shows the results. On the LOX-1 immobilized plate, the binding of LOX-1 to FV was significantly inhibited in the wells to which the anti-LOX-1 antibodies were added (there is a significant difference at a level of significance of less than 5%), as compared to the well to which FV alone was added and the well to which FV and IgG were added. Similarly, the binding of LOX-1 to FVa was significantly inhibited by the anti-LOX-1 antibodies.

### Example 4

### Determination of LOX-1 Binding Site of FVa Using Anti-L Chain Antibody and Anti-H Chain Antibody

Which site of FVa LOX-1 binds to was determined by ELISA. ELISA was performed in the following manner. On a 384-well plate (manufactured by Greiner), LOX-1 (5 μg/mL for each well) was immobilized at 4° C. overnight. After blocked with a solution containing 3% BSA/10 mM HEPES and 150 mM NaCl at pH7.4, the plate was washed twice with PBS (−). FV or FVa was added to the plate and was then reacted for 1 hour at room temperature. Thereafter, an anti-L chain antibody was added, a 2 mM CaCl₂ solution or 2 mM EDTA was further added, and the plate was then incubated for 1 hour at room temperature. Then, absorbance at 450 nm was measured. FVa was added at the concentration of 0 μg/mL, 0.03 μg/mL, 0.1 μg/mL, 0.3 μg/mL, 1 μg/mL, or 3 μg/mL. Further, controls using BSA instead of LOX-1 were also measured in a similar manner. Still further, the anti-L chain antibody was replaced by the anti-H chain antibody, and measurement was then performed in a similar manner. Anti-Human Factor V: AHV-5101, purchased from Haematologic Technologies Inc. was used as the anti-L chain antibody, while Anti-Human Factor V: AHV-5146, purchased from Haematologic Technologies Inc., was used as the anti-H chain antibody.

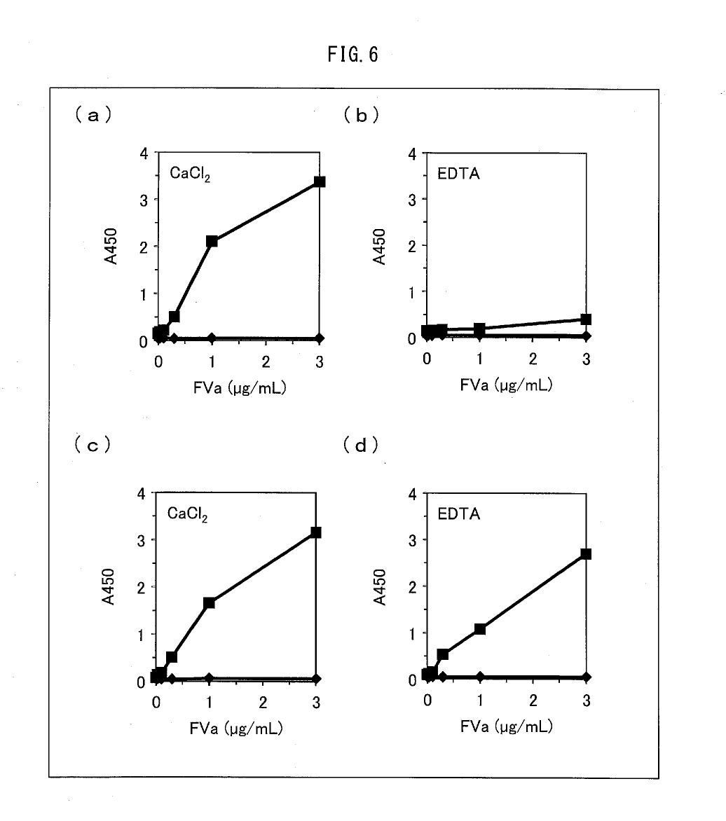

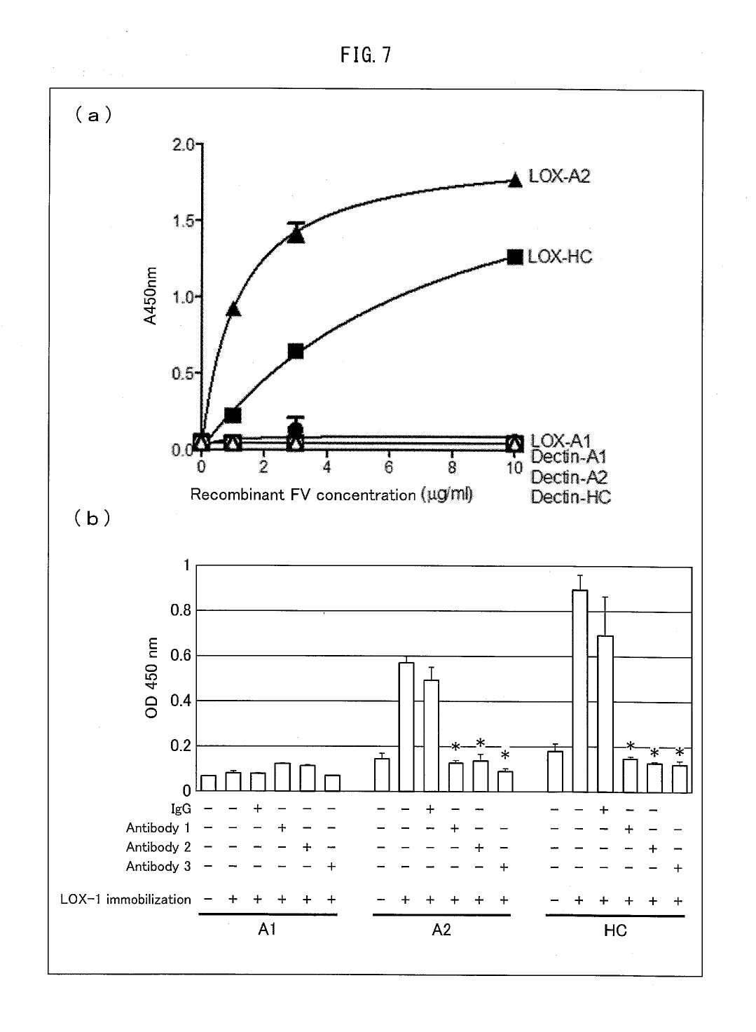

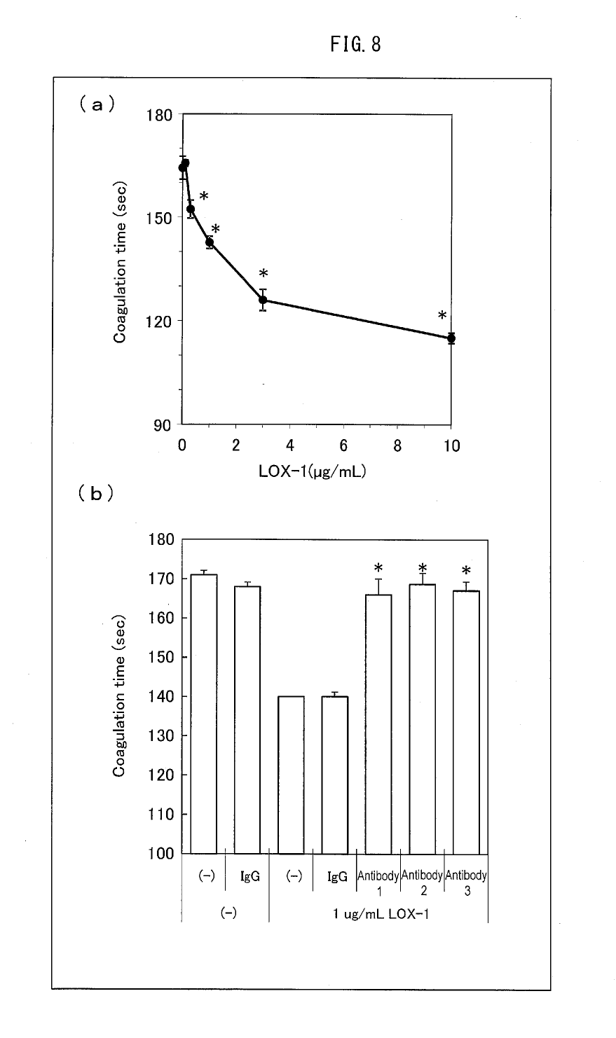

FIG. 6 shows results of ELISA. (a) of FIG. 6 shows a result obtained for the well to which the anti-L chain antibody and the CaCl₂ solution were added, and (b) of FIG. 6 shows a result obtained for the well to which the anti-L chain antibody and EDTA were added. (c) of FIG. 6 shows a result obtained for the well to which the anti-H chain antibody and the CaCl₂ solution were added, and (d) of FIG. 6 shows a result obtained for the well to which the anti-H chain antibody and EDTA were added.

In the presence of Ca²⁺, the H chain and the L chain were integrated in a manner shown in FIG. 2. In the well treated with EDTA, the binding of the H chain and the L chain disappears. The binding of LOX-1 to FVa was detected by the anti-L chain antibody as shown in (a) of FIG. 6, but almost no binding of LOX-1 to FVa was detected in the EDTA-treated well as shown in (b) of FIG. 6. On the other hand, the binding of LOX-1 to FVa was detected by the anti-H chain antibody as shown in (c) of FIG. 6, and was also detected in much the same degree even in the EDTA-treated well, as shown in (d) of FIG. 6. This proves that LOX-1 binds to the H chain of FVa.

### Example 5

### Binding of LOX-1 to Each Domain of H Chain of FV

Which site of the H chain of FV LOX-1 binds to was determined by ELISA. ELISA was performed in the following manner. FV as used was a recombinant FV prepared by the following method. First, FV H-chain plasmid (containing NM000130.4 (1-709, aa), pcDNA3.3, V5-6×His, and Igκ secretion signal therein), FV A1 protein plasmid (containing NM000130.4 (1-303, aa), pEF6/V5-His, and Igκ secretion signal therein), or FV A2 protein plasmid (containing NM000130.4 (317-656, aa), pcDNA3.3, V5-6×His, and Igκ secretion signal therein) was transfected into FreeStyle293 cells. Note that the Igκ secretion signal was obtained by pSecTag/FRT/V5-HisTOPO (Invitrogen). Subsequently, H chains, A1, and A2 of FV secreted by the FreeStyle293 cells during the culture were collected and then prepared by His-tag purification with TALON Metal Affinity Resin (TAKARA).

ELISA was performed in the following manner. On a 384-well plate (manufactured by Greiner), LOX-1 (5 μg/mL) was immobilized at 4° C. overnight. After blocked with a solution containing 3% BSA/10 mM HEPES and 150 mM NaCl at pH7.4, the plate was washed twice with PBS (−). To the plate, the H chain of FV, A1 domain of the H chain of FV, or A2 domain of the H chain of FV, was added at 0 μg/mL, 1 μg/mL, 3 μg/mL, or 10 μg/mL, and was then reacted for 1 hour at room temperature. Detection was performed with use of Horseradish peroxidase streptavidin-labeled anti-V5 antibody and a TMB reagent. Then, absorbance at 450 nm was measured. Controls using Dectin-1 instead of LOX-1 were also measured in a similar manner.

(a) of FIG. 7 shows the results. Binding of LOX-1 to the H chain of FV and binding of LOX-1 to the A2 domain of the H chain of FV were detected. Binding of LOX-1 to the A1 domain was not detected. This proves that LOX-1 binds to the A2 domain of the H chain of FV.

### Example 6

### Inhibitory Effect of Anti-LOX-1 Antibody on Binding of LOX-1 to A2 Domain of H Chain of FV

Further, it was confirmed by ELISA that the binding of LOX-1 to the A2 domain of the H chain of FV is suppressed by the anti-LOX-1 antibodies. As in Example 5, a recombinant FV was used as the FV. As in Example 3, HUC348 (Antibody 1), HUC52 (Antibody 2), and HUC52 humanized antibody (Antibody 3) were used as the anti-LOX-1 antibodies.

ELISA was performed in the following manner. On a 384-well plate (manufactured by Greiner), LOX-1 (5 μg/mL) was immobilized at 4° C. overnight. After blocked with a solution containing 3% BSA/10 mM HEPES and 150 mM NaCl at pH7.4, the plate was washed twice with PBS (−). To the plate were added 10 μg/mL of anti-LOX-1 antibodies or IgG, after which the plate was pre-incubated. The plate was washed twice with PBS (−), and 3 μg/mL of H chain of FV, 10 μg/mL of A1 domain of H chain of FV, or 10 μg/mL of A2 domain of H chain of FV was added to each well, and was then reacted for 1 hour at room temperature. Detection was performed with use of anti-V5 antibodies, biotin-labeled anti-mouse IgG antibodies, Horseradish peroxidase streptavidin, and a TMB reagent. Then, absorbance at 450 nm was measured. A control in which LOX-1 was not immobilized and a control to which neither IgG nor any anti-LOX-1 antibody were added, were also measured in a similar manner.

(b) of FIG. 7 shows the results. From the control to which neither IgG nor anti-LOX-1 antibody were added, the binding of FV was detected at the H chain and A2 domain. The binding of FV was also detected similarly from the well to which IgG was added. On the other hand, the binding of FV (H chain and A2 domain) was significantly inhibited in the wells to which the anti-LOX-1 antibodies were added (there is a significant difference at a level of significance of less than 5%).

### Example 7

### Anti-Blood Coagulation Effect Yielded by the Anti-LOX-1 Antibody

An anti-blood coagulation effect yielded by the anti-LOX-1 antibody was confirmed by the recalcification blood coagulation test. To a glass test tube, recombinant LOX-1 protein was added at the concentration of 0 μg/mL, 0.1 μg/mL, 0.3 μg/mL, 1 μg/mL, 3 μg/mL, or 10 μg/mL. The recombinant LOX-1 protein was obtained by the same method as that in Example 2. Further, human blood plasma obtained by using a citric acid was added to the test tube. Thereafter, Ca²⁺ was added to the test tube at a final concentration of 12.5 mM. While the test tube was slowly shaken in a hot water bath at 37° C., fluidity was observed. A time elapsed until the mixture in the test tube turned into a clot which does not drop out of the reversed test tube was measured as a coagulation time. Then, the coagulation times of test tubes to which (i) 1 μg/mL of recombinant LOX-1 protein and (ii) IgG or each anti-LOX-1 antibody were added, were also measured in a similar manner. Furthermore, the coagulation time of a test tube to which no recombinant LOX-1 protein was added, was also measured in a similar manner. As in Example 3, HUC348 (Antibody 1), HUC52 (Antibody 2), and HUC52 humanized antibody (Antibody 3) were used as the anti-LOX-1 antibodies.

(a) of FIG. 8 shows the results obtained for the test tubes to which neither IgG nor any anti-LOX-1 antibody were added and shows coagulation times that vary depending on the concentration of LOX-1. The coagulation time was reduced in a manner dependent on the concentration of the added recombinant LOX-1 protein. Especially, the coagulation time of the test tube to which the recombinant LOX-1 protein was added in an amount of 10 μg/mL was reduced to two-thirds of the coagulation time of the test tube in which the recombinant LOX-1 protein was 0 μg/mL. This proves that the addition of LOX-1 significantly reduces a blood coagulation time (there is a significant difference at a level of significance of less than 5%).

(b) of FIG. 8 shows the results obtained for the test tubes to which IgG or the anti-LOX-1 antibodies were added. First, IgG, when added to the blood plasma alone, had no influence on the coagulation time. In the case of the test tube in which LOX-1 was added to the blood plasma, the coagulation time was reduced. In the case of the test tube in which IgG was added to the mixture of LOX-1 and the blood plasma, no difference was observed in the coagulation time from the test tube in which LOX-1 alone was added to the blood plasma. On the other hand, in the case of the test tube in which the anti-LOX-1 antibody was added to the mixture of LOX-1 and the blood plasma, it was observed that the coagulation time was on a par with the coagulation time of the test tube in which no LOX-1 was added. Note that, in the case of the test tube to which both LOX-1 and the anti-LOX-1 antibody were added, the coagulation time significantly increased (there is a significant difference at a level of significance of less than 5%), as compared to the coagulation time of the test tube in which LOX-1 alone was added to the blood plasma. That is, it is determined that the anti-LOX-1 antibody has no influence on the coagulation reaction which is independent of LOX-1, but suppresses only a coagulation reaction which is promoted by LOX-1.

### Example 8

### Coagulation Reaction in FV-Deficient Blood Plasma

A coagulation reaction in FV-deficient blood plasma was confirmed by the recalcification blood coagulation test. Recombinant LOX-1 protein was added to a plastic test tube at the concentration of 10 μg/mL and was then immobilized. To the test tube, FV or FVa was added at the concentration of 1 μg/mL. FV and FVa not binding to the LOX-1 protein were washed off. Further, the FV-deficient blood plasma was added to the test tube. Thereafter, Ca²⁺ was added to the test tube at a final concentration of 12.5 mM, and the coagulation time was measured as in Example 7. Further, the coagulation times were also measured in a similar manner for a test tube to which Dectin-1 was added instead of LOX-1 and for a test tube to which neither LOX-1 nor Dectin-1 were added.

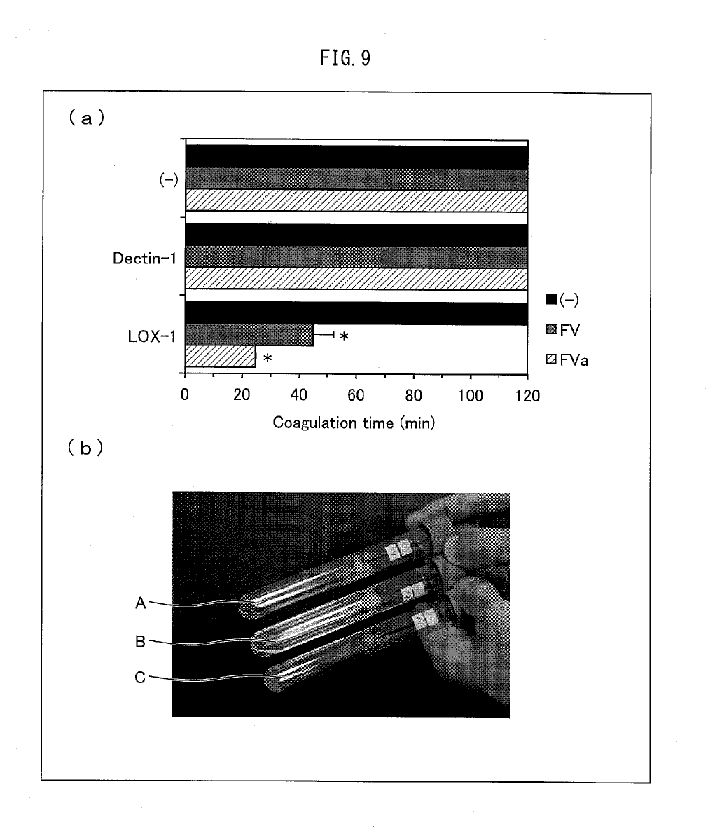

(a) and (b) of FIG. 9 are views showing the results of coagulation reactions in the FV-deficient blood plasma. As shown in (a) of FIG. 9, the coagulation time was reduced only in the case of the test tube in which FV or FVa was bound to LOX-1 (there is a significant difference at a level of significance of less than 5%). (b) of FIG. 9 shows the results obtained for the test tube A in which neither LOX-1 nor Dectin-1 were added, for the test tube B in which Dectin-1 was added, and for the test tube C in which LOX-1 was added, wherein FV was added to each of the test tubes A, B, and C. As is also clear from (b) of FIG. 9, a coagulation occurred only in the case of the test tube in which LOX-1 was bound to FV. This proves that the binding of LOX-1 to FV or FVa causes a coagulation reaction.

### Example 9

### Anti-Blood Coagulation Effect Yielded by Procyanidin

An anti-blood coagulation effect yielded by procyanidin was confirmed by the recalcification blood coagulation test. To a glass test tube, 1 μg/mL of recombinant LOX-1 protein and 1 μg/mL of procyanidin were added. Thereafter, as in Example 8, human blood plasma and Ca²⁺ were added to the test tube, and the coagulation time was measured. Then, the coagulation time of the test tube to which no procyanidin was added, was also measured in a similar manner. Furthermore, the coagulation time of the test tube to which no recombinant LOX-1 protein was added, was also measured in a similar manner.

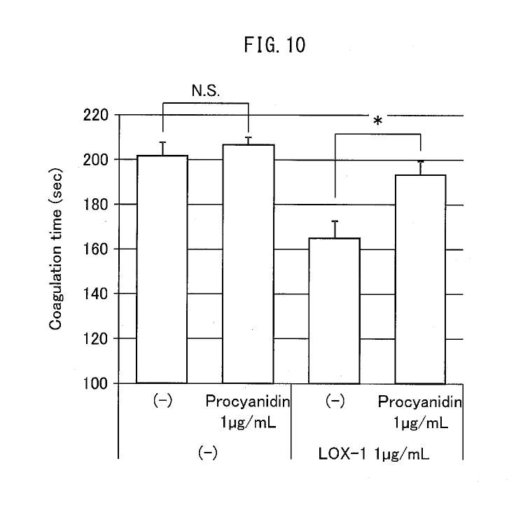

FIG. 10 shows the results. Procyanidin, when added to the blood plasma alone, had no influence on the coagulation time. In the case of the test tube in which LOX-1 was added to the blood plasma, the coagulation time was reduced. In the case of the test tube in which procyanidin was added to the mixture of LOX-1 and the blood plasma, it was observed that reduction of the coagulation time was suppressed (there is a significant difference at a level of significance of less than 5%). That is, it is determined that procyanidin has no influence on the coagulation reaction which is independent of LOX-1, but suppresses only a coagulation reaction which is promoted by LOX-1.

### INDUSTRIAL APPLICABILITY

As described above, the present invention provides an anti-blood coagulant that selectively suppresses a pathological blood coagulation reaction without increasing the risk of major bleeding. As such, the anti-blood coagulant in accordance with the present invention serves as effective means for treating or preventing thrombosis and other diseases for which LOX-1 is responsible.

Therefore, the present invention is applicable in medical and pharmaceutical industries.

1. An anti-blood coagulant comprising:
a binding inhibiting component that inhibits binding of LOX-1 to at least one of factor V and factor Va.

2. The anti-blood coagulant according to claim 1, wherein the binding inhibiting component is a binding inhibiting component that inhibits binding of LOX-1 to an H chain of at least one of factor V and factor Va.

3. The anti-blood coagulant according to claim 2, wherein the binding inhibiting component is a binding inhibiting component that inhibits binding of LOX-1 to A2 domain of at least one of factor V and factor Va.

4. The anti-blood coagulant according to any one of claim 1, wherein the binding inhibiting component is an anti-LOX-1 antibody.

5. The anti-blood coagulant according to any one of claim 1, wherein the binding inhibiting component is polyphenol.

6. A pharmaceutical composition comprising the anti-blood coagulant according to any one of claim 1.


SEQUENCE LISTING (8 sequences)

SEQ ID NO: 1 - protein, 13 aa, Homo sapiens
RAVQPGETYTYKW

SEQ ID NO: 2 - protein, 12 aa, Homo sapiens
RLLSLGAGEFRS

SEQ ID NO: 3 - protein, 10 aa, Homo sapiens
KSQFLIKTRK

SEQ ID NO: 4 - protein, 20 aa, Homo sapiens
RETDIEDSDDIPEDTTYKKV

SEQ ID NO: 5 - protein, 11 aa, Homo sapiens
RKYLDSTFTKR

SEQ ID NO: 6 - protein, 14 aa, Homo sapiens
RAEVDDVIQVRFKN

SEQ ID NO: 7 - protein, 17 aa, Homo sapiens
RAGMQTPFLIMDRDCRM

SEQ ID NO: 8 - protein, 17 aa, Homo sapiens
KSSMVDKIFEGNTNTKG